(19) United States
(12) Patent Application Publication
(10) Pub. No.: US 2026/0367264 A1
(43) Pub. Date: Nov. 19, 2026

(54) METHOD AND SYSTEM FOR INTELLIGENT IDENTIFICATION OF PARTICLE TYPES PRODUCED BY MEDIUM-LOW ENERGY HEAVY ION NUCLEAR REACTIONS

(71) Applicant: SHANGHAI UNIVERSITY OF ENGINEERING SCIENCE, Shanghai (CN)

(72) Inventors: Qianmin SU, Shanghai (CN); Gaoyi CHENG, Shanghai (CN); Lihong KONG, Shanghai (CN)

(21) Appl. No.: 19/161,916

(22) PCT Filed: Jul. 8, 2024
(86) PCT No.: PCT/CN2024/104289

§ 371 Date: Sep. 3, 2025

(30) Foreign Application Priority Data
Feb. 2, 2024 (CN) 202410147182.9

(51) Int. Cl. G01T 1/36; G06N 3/126; G16C 20/10; G16C 20/70

(52) U.S. Cl. CPC G01T 1/36; G06N 3/126; G16C 20/10; G16C 20/70

(57) ABSTRACT

The present invention discloses a method and system for intelligent identification of particle types produced by medium-low energy heavy ion nuclear reactions. The method includes: creating a particle library containing all possible particle types that can be produced by current medium-low energy heavy ion nuclear reactions; constructing a particle identification curve set using a genetic algorithm and the created particle library; collecting the input original data of two-dimensional energy spectrum characteristics E and ΔE of the particles to be identified; and using the constructed particle identification curve set to identify the particle types. This invention not only achieves intelligent identification of particle types produced by medium-low energy heavy ion nuclear reactions, and solves the problems inherent in the existing manual particle identification methods, such as time-consuming, labor-intensive, inefficient, poor reproducibility, low accuracy, high workload, and difficulty in dealing with a large amount of complex experimental data, but also supports use in various experimental environments and online environments. It offers advantages such as speed, convenience, high efficiency, and high accuracy, making it of significant application value in nuclear physics research.

### TECHNICAL FIELD

The present invention relates to a method and system for intelligent identification of particle types produced by medium-low energy heavy ion nuclear reactions, and belongs to the field of information processing technologies.

### BACKGROUND OF THE INVENTION

Heavy ions refer to atomic nuclei with a mass number or charge number greater than that of an alpha particle. The collision between heavy ions and atomic nuclei is called a heavy ion nuclear reaction. Medium-low energy heavy ion nuclear reactions refer to reactions occurring in a medium energy range. These reactions are currently the only method available in laboratory experiments on Earth to study the properties of high-temperature, high-density nuclear matter. Such reactions can be used to explore the fundamental structure of matter and understand the origin of the universe, making them highly significant in physical research.

The main task of particle identification is to confirm the charge number and mass number of the particles produced in nuclear reactions, helping researchers identify the particle types, thereby understanding the nuclear reaction process and further studying the results of the reaction. In heavy ion nuclear reaction experiments, particle identification is a critical step. Only by accurately identifying the different types of particles can their properties and interactions be effectively studied.

In medium-low energy heavy ion nuclear reaction experiments, researchers typically use telescope-type detectors to collect particle information produced during nuclear reactions. These detectors consist of a set of thin scintillation detectors and a thicker scintillation detector. By plotting the deposited energy ΔE (from the thin scintillator) on the y-axis and the deposited energy E (from the thick scintillator) on the x-axis, a two-dimensional histogram of the particle's energy spectrum (also called a two-dimensional energy spectrum) can be constructed. In the two-dimensional energy spectrum, the scatter points of the particles form multiple hyperbolic particle clusters, where particles within the same band cluster have the same charge number and mass number.

Currently, in the field of nuclear physics, the task of identifying particle types mainly relies on manual methods. Researchers use professional nuclear data analysis software to manually cut, fit, and linearize the two-dimensional energy spectrum of the particles, and then manually judge the particle type based on the peaks of the linearized one-dimensional spectrum. This identification method suffers from issues such as poor repeatability, high time consumption, low efficiency, high workload, and is particularly problematic because, with the advancement of scientific theories and continuous improvements in experimental instruments, the volume of data produced by medium-low energy heavy ion nuclear reactions has reached the scale of billions. If the current manual identification method is used, it is no longer capable of handling the complex experimental demands and the need for rapid identification. Therefore, the field urgently needs a technology that can intelligently identify the particle types produced by medium-low energy heavy ion nuclear reactions.

### SUMMARY OF THE INVENTION

In view of the above-described problems and needs in the prior art, it is an object of the present invention to provide a method and system for intelligent identification of particle types produced by medium-low energy heavy ion nuclear reactions.

To this end, the present invention lies in the following technical solution:

A method for intelligent identification of particle types produced by medium-low energy heavy ion nuclear reactions, comprising the following steps:
S1) Create a particle library containing all possible particle types that can be produced by current medium-low energy heavy ion nuclear reactions, where the particle library is a nested array consisting of arrays representing particle types by their charge number and mass number;
S2) Use a genetic algorithm and the particle library created in step S1) to construct a set of particle identification curves, where the particle identification curves conform to the following formula (1):

$$Δ E = {[ {( g E )}^{μ + v + 1} + {( λ {Z}^{α} {A}^{β} )}^{μ + v + 1} + ξ {Z}^{2} {{A}^{μ} ( g E )}^{v} ]}^{( \frac{1}{μ + v + 1} )} - gE ; \qquad ( 1 )$$

Where: E and ΔE are the two-dimensional energy spectrum feature values of the particles, Z is the charge number of the particle, A is the mass number of the particle, and g, λ, ζ, α, β, μ and v are the parameters of the particle identification curve, all the parameters of the particle identification curve are greater than 0 and are iteratively optimized using the genetic algorithm;
The particle identification curve set is a collection of multiple particle identification curves formed by substituting the particle identification curve parameters and their corresponding particle type charge numbers and mass numbers into formula (1);
S3) Clean the input data of the particles to be identified that are produced by current medium-low energy heavy ion nuclear reactions, and extract only the collected two-dimensional energy spectrum feature data E and ΔE for each particle;
S4) Substitute the collected data of E corresponding to the particle j to be identified into formula (1) described in step S2), and sequentially calculate the ΔE values using the particle identification curve parameters and charge number and mass number corresponding to each particle identification curve in the particle identification curve set constructed in step S2), and denote the calculated ΔE value as ΔEⱼ; The absolute difference between ΔE (the original collected value) and ΔEⱼ (the calculated value) is taken as the distance from the particle j to the corresponding particle identification curve, and the charge number and mass number of the particle identification curve that results in the minimum absolute value of ΔE−ΔEⱼ are determined to be the type for particle j.

Further, the process of constructing the particle identification curve set in step S2) includes the following specific steps:
S21) Obtain an existing particle dataset for a reaction identical to the current medium-low energy heavy ion nuclear reaction, clean the data to remove redundant features, and extract the required two-dimensional energy spectrum feature data E and ΔE for each particle;
S22) Randomly generate a certain number of individuals, whose collection is referred to as the population; Each individual is composed of the particle identification curve parameters g, λ, ζ, α, β, μ, v and an integer variable Nᵢ to determine whether the particle type in the particle library exists in the obtained existing dataset, the value of i corresponds to the sequence number of the particle type in the particle library, starting from 1, and the value of Nᵢ is either 0 or 1; When Nᵢ is 0, it means the particle type does not exist in the dataset; When Nᵢ is 1, it means the particle type exists in the dataset; The initial values of g, λ, ζ, α, β, μ, v and Nᵢ for each individual are all randomly generated by the system;
S23) Calculate the fitness value y for each individual using the fitness function shown in the following formula (2):

$$y = 100 * \frac{1}{N l} * {\sum }_{i = 1}^{Nl} \frac{{\sum }_{j = 1}^{{Np}_{i}} {( Δ E - Δ {E}_{j} )}^{2}}{{Np}_{i}} \qquad ( 2 )$$

Where: Nl represents the number of curves, Npᵢ represents the number of particles corresponding to the i-th curve, ΔEⱼ represents the ΔE calculated for particle j using formula (1), and ΔE represents the original collected value for particle j; Additionally, Npᵢ is not 0, Nl refers to the number of curves corresponding to particle type where Nᵢ is 1 and Npᵢ is not 0; The value of ΔE−ΔEⱼ is the minimum distance between particle j and the curves corresponding to particle type where Nᵢ is 1;
S24) Based on the calculated fitness value y for each individual, select suitable individuals from the population to generate the next generation;
S25) Add the generated next generation to the population by replacement or direct addition;
S26) Repeat steps S21) to S25) until the iteration stopping conditions are met;
S27) After the iteration is completed, output the individual with the smallest fitness value y;
S28) Combine the charge number and mass number of the particle type where Nᵢ=1 and Npᵢ≠0 with the particle identification curve parameters g, λ, ζ, α, β, μ and v from the output individual, and substitute them into formula (1) to obtain the particle identification curve set.

As an optional solution, the method further includes the following optimization iteration step:
S29) Group the particles in the constructed particle identification curve set by charge number, and group particles with the same charge number together; then, perform steps S21) to S28) for each group of particles to optimize the iteration.

An example of the method described in step S23) includes the following specific steps:
S231) Traverse the existing particle dataset obtained in step S21);
S232) Obtain the two-dimensional energy spectrum feature data E and ΔE for particle j;
S233) For each individual, sequentially calculate the values of ΔE−ΔEⱼ using the particle identification curve parameters and the charge number and mass number of the particle type where Nᵢ is 1, select the minimum absolute value and increment Npi for the particle type corresponding to the smallest absolute value to count the number of particles of that type;
S234) Continue to obtain the next particle's two-dimensional energy spectrum feature data E and ΔE and repeat step S233).

An example of step S24) includes the following specific steps:
S241) Selection: Based on the calculated fitness value y for each individual, select individuals with relatively smaller fitness values as the next generation;
S242) Mutation: Randomly modify the data features of some individuals in the population to prevent excessive similarity between individuals, thus promoting population diversity;
S243) Crossover: Perform random crossover combinations of some individuals in the population to generate new individuals.

An example of the iteration stopping condition in step S26) is a pre-set maximum number of iterations, a continuous stagnation limit, or a fitness threshold.

An example of the method further includes the following step:
S5) Save the outlier particle data whose distance to every particle identification curve in the particle identification curve set exceeds the preset threshold; After reaching the preset quantity, re-execute Step S2) with the original data to update the particle identification curve set.

A system for intelligent identification of particle types produced by medium-low energy heavy ion nuclear reactions, comprising:
Data processing module, which is used for cleaning and processing the raw data of the particles produced by medium-low energy heavy ion nuclear reactions, in order to extract the two-dimensional energy spectrum features E and ΔE of the particles;
Particle identification curve set construction module, which is used to create a particle library and construct a particle identification curve set using a genetic algorithm;
Particle type identification module, which is used to identify the particle types produced by medium-low energy heavy ion nuclear reactions.

Additionally, the present invention provides a storage medium, wherein the storage medium stores one or more computer programs including executable instructions, which can be read and executed by an electronic device (including but not limited to a computer, server, or network device) to implement the method for intelligent identification of particle types produced by medium-low energy heavy ion nuclear reactions as described in the present invention.

The present invention also provides an electronic device, which comprising:
at least one processor; and
a memory communicatively coupled to the at least one processor and storing program instructions executable by the at least one processor;
wherein the program instructions, when executed by the at least one processor, can cause the at least one processor to perform the method for intelligent identification of particle types produced by medium-low energy heavy ion nuclear reactions as described in the present invention.

The present invention further provides a computer program product, comprising a computer program stored on a storage medium, the computer program comprising program instructions, wherein, when the program instructions are executed by a computer, they can implement the method for intelligent identification of particle types produced by medium-low energy heavy ion nuclear reactions as described in the present invention.

Compared to the prior art, the beneficial technical effects of the present invention are as follows:

The present invention first creates a particle library containing all possible particle types produced by medium-low energy heavy ion nuclear reactions, based on existing nuclear physics research information. This library, combined with the collected particle two-dimensional deposition energy characteristic data, is used to construct a particle identification curve set using a genetic algorithm. The particle type for each particle to be identified is then determined by calculating the distance between the particle and each particle identification curve in the set, and by judging whether the distance is within a preset threshold range and which particle identification curve has the closest distance. This not only achieves intelligent identification of particle types produced by medium-low energy heavy ion nuclear reactions, but also solves the problems inherent in the existing manual particle identification methods, such as time consumption, labor intensity, low efficiency, poor repeatability, low accuracy, high work intensity, and difficulty in dealing with a large amount of complex experimental data. Furthermore, the method does not require prior data labeling or training any data models. It supports usage in various experimental environments, including online environments, offering advantages of speed, convenience, high efficiency, and high accuracy, making it of significant application value in nuclear physics research.

### BRIEF DESCRIPTION OF THE DRAWINGS

FIG. 1 is a flowchart illustrating the implementation of the method provided by an embodiment of the present invention for intelligent identification of particle types produced by medium-low energy heavy ion nuclear reactions.

FIG. 2 is a flowchart of the construction process of the particle identification curve set described in the embodiment of the present invention.

FIG. 3 is a visualization diagram of the particle identification curve set constructed in the embodiment of the present invention.

FIG. 4 is a flowchart illustrating the steps executed by computer for the method of intelligent identification of particle types produced by medium-low energy heavy ion nuclear reactions according to the present invention.

FIG. 5 is a structural block diagram of a system provided by an embodiment of the present invention for intelligent identification of particle types produced by medium-low energy heavy ion nuclear reactions.

### DETAILED DESCRIPTION OF THE INVENTION

The technical solution of the present invention will be further described in detail and in full with reference to the examples and the accompanying drawings.

### Example

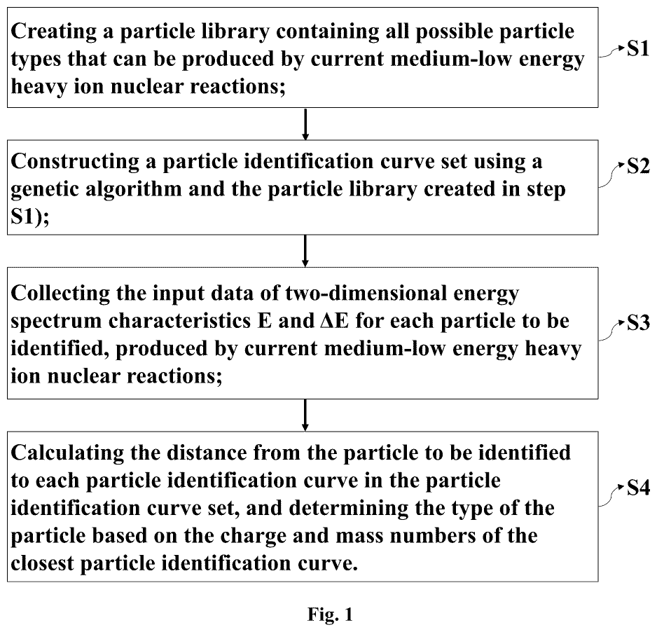

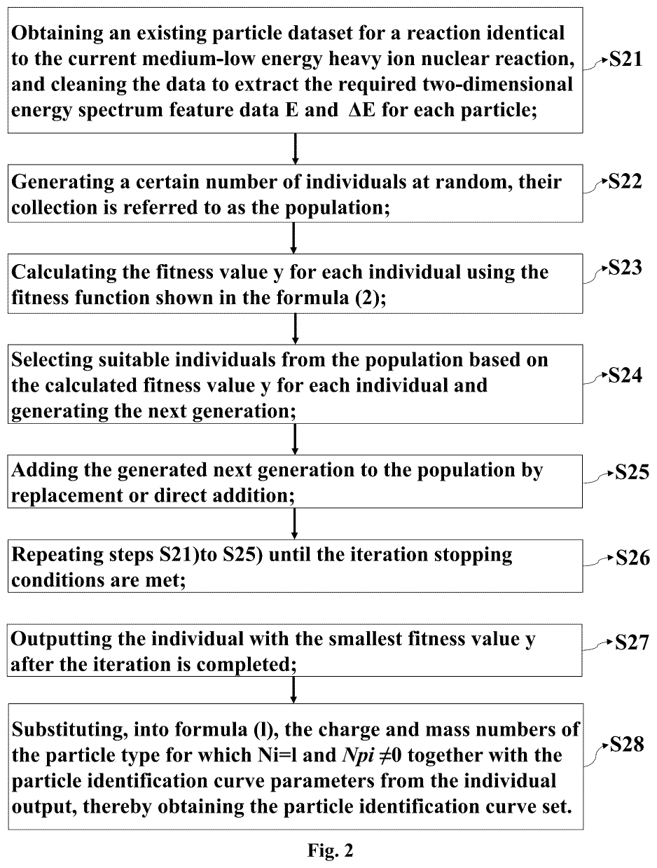

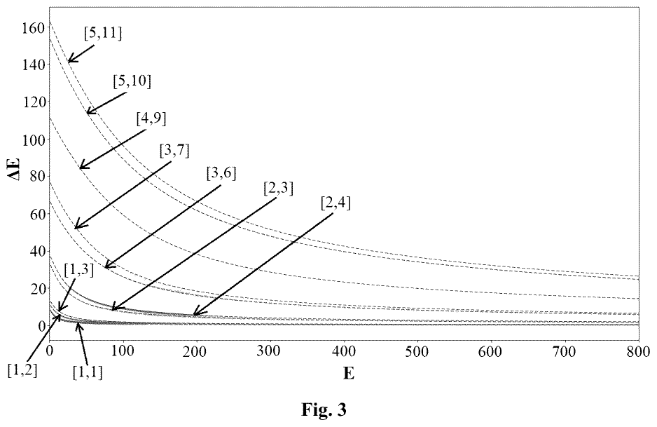

With reference to FIG. 1 and FIG. 2, a method for intelligent identification of particle types produced by medium-low energy heavy ion nuclear reactions includes the following steps:
S1) Create a particle library containing all possible particle types that can be produced by current medium-low energy heavy ion nuclear reactions, where the particle library is a nested array consisting of arrays representing particle types by their charge number and mass number; For example, the particle library constructed in this example is [[1, 1], [1, 2], [1, 3], [2, 3], [2, 4], [3, 6], [3, 7], [4, 9], [5, 10], [5, 11]], which is a nested array composed of ten particle type arrays; The particle library is initially created based on known nuclear physics research information, and can be continuously updated as research progresses;
S2) Use a genetic algorithm and the particle library created in step S1) to construct a set of particle identification curves, where the particle identification curves conform to the following formula (1):

$$ΔE = {[ {( gE )}^{μ + v + 1} + {( λ {Z}^{α} {A}^{β} )}^{μ + v + 1} + ξ {Z}^{2} {{A}^{μ} ( g E )}^{v} ]}^{( \frac{1}{μ + v + 1} )} - gE ; \qquad ( 1 )$$

Where: E and ΔE are the two-dimensional energy spectrum feature values of the particles, Z is the charge number of the particle, A is the mass number of the particle, and g, λ, ζ, α, β, μ and v are the parameters of the particle identification curve, all the parameters of the particle identification curve are greater than 0 and are iteratively optimized using the genetic algorithm;
The particle identification curve set is a collection of multiple particle identification curves formed by substituting the particle identification curve parameters and their corresponding particle type charge numbers and mass numbers into formula (1);
The specific construction process includes the following steps (please refer to FIG. 2):
S21) Obtain an existing particle dataset for a reaction identical to the current medium-low energy heavy ion nuclear reaction, clean the data to remove redundant features, and extract the required two-dimensional energy spectrum feature data E and ΔE for each particle; For example, in this example, an existing particle dataset consisting of a total of 21,248 particles is obtained, among them, there are 14,224 particles with a charge number of 1, there are 5,629 particles with a charge number of 2, there are 735 particles with a charge number of 3, there are 180 particles with a charge number of 4, and there are 480 particles with a charge number of 5;
S22) Randomly generate a certain number of individuals, whose collection is referred to as the population; Each individual is composed of the particle identification curve parameters g, λ, ζ, α, β, μ, y and an integer variable Nᵢ to determine whether the particle type in the particle library exists in the obtained existing dataset, the value of i corresponds to the sequence number of the particle type in the particle library, starting from 1, for example, Nᵢ represents the particle type [1, 1] in the first position of the particle library, and N₆ represents the particle type [3, 6] in the sixth position of the particle library; And the value of Nᵢ is either 0 or 1; When Nᵢ is 0, it means the particle type does not exist in the dataset; When Nᵢ is 1, it means the particle type exists in the dataset; for example, if Nᵢ is 0, it indicates that the particle type [1, 1] in the first position of the particle library does not exist in the obtained existing particle dataset; If N₃ is 1, it indicates that the particle type [1, 3] in the third position of the particle library exists in the obtained existing particle dataset; The initial values of g, λ, ζ, α, β, μ, v and Nᵢ for each individual are all randomly generated by the system;
S23) Calculate the fitness value y for each individual using the fitness function shown in the following formula (2):

$$y = 10 \; 0 * \frac{1}{N l} * {\sum }_{i = 1}^{Nl} \frac{{\sum }_{j = 1}^{{Np}_{i}} {( Δ E - Δ {E}_{j} )}^{2}}{{Np}_{i}} ; \qquad ( 2 )$$

Where: Nl represents the number of curves, Npᵢ represents the number of particles corresponding to the i-th curve, ΔEⱼ represents the ΔE calculated for particle j using formula (1), and ΔE represents the original collected value for particle j; Additionally, Npᵢ is not 0, Nl refers to the number of curves corresponding to particle type where Nᵢ is 1 and Npᵢ is not 0; The value of ΔE−ΔE, is the minimum distance between particle j and the curves corresponding to particle type where Nᵢ is 1;
The calculation of the fitness value y for each individual includes the following steps:
S231) Traverse the existing particle dataset obtained in step S21);
S232) Obtain the two-dimensional energy spectrum feature data E and ΔE for particle j;
S233) For each individual, sequentially calculate the values of ΔE−ΔEⱼ using the particle identification curve parameters and the charge number and mass number of the particle type where Nᵢ is 1, select the minimum absolute value and increment Npi for the particle type corresponding to the smallest absolute value to count the number of particles of that type;
S234) Continue to obtain the next particle's two-dimensional energy spectrum feature data E and ΔE and repeat step S233);
S24) Based on the calculated fitness value y for each individual, select suitable individuals from the population to generate the next generation; this process includes the following steps:
S241) Selection: Based on the calculated fitness value y for each individual, select individuals with relatively smaller fitness values as the next generation; The core of the selection strategy is survival of the fittest, where individuals with smaller fitness values replace those with larger fitness values; The selection algorithm can use any one of the following: Boltzmann, random traversal, roulette, tournament, or Monte Carlo algorithms;
S242) Mutation: Randomly modify the data features of some individuals in the population to prevent excessive similarity between individuals, thus promoting population diversity; The mutation algorithm can use any one of the following: bit mutation, random reset, exchange mutation, or uniform mutation;
S243) Crossover: Perform random crossover combinations of some individuals in the population to generate new individuals; The core of crossover is to use parent individuals to create one or more new offspring individuals; The crossover algorithm can use any one of the following: single-point crossover, multi-point crossover, uniform crossover, or random crossover;
S25) Add the generated next generation to the population by substitution or direct addition;
S26) Repeat steps S21) to S25) until the iteration stopping conditions are met; The iteration stopping conditions are the pre-set maximum iteration count, the upper limit of continuous stagnation, or the fitness threshold; In this example, the individual in the genetic algorithm is set as a 1*17 variable, the maximum iteration count is set to 2000, and the maximum upper limit of the evolution stagnation counter is set to 20; The genetic algorithm is implemented using the soea_SEGA template interface provided by GEATpy, it allows the types of decision variables to be specified: the first seven parameters (particle identification curve parameters) are declared as floating-point variables, while the remaining ten parameters are declared as integer variables;
S27) After the iteration is completed, output the individual with the smallest fitness value y; In this example, after the iteration is finished, the output is [1.067, 0.815, 0.1, 2.9684, 1.009, 0.5242, 58.908, 1, 1, 1, 1, 1, 1, 1, 1, 1, 1], where the first 7 parameters correspond to the particle identification curve parameters g, λ, ζ, α, β, μ and v in formula (1), and the last 10 parameters indicate that all 10 particle type in the particle library exist in the existing particle dataset obtained in step S21);
S28) Combine the charge number and mass number of the particle type where Nᵢ=1 and Npᵢ≠0 with the particle identification curve parameters g, λ, ζ, α, β, μ and v from the output individual, and substitute them into formula (1) to obtain the particle identification curve set;
S29) Group the particles in the constructed particle identification curve set by their charge number, with particles having the same charge number being grouped together, then, for each group, repeat steps S21) to S28) for optimization iteration;
In this example, the number of particles corresponding to each particle type in the obtained existing particle dataset is [10671, 2678, 875, 867, 4762, 525, 210, 180, 297, 183], and since there are 5 charge numbers, the particles are grouped into 5 groups, specifically:
For particles with a charge number of 1 (corresponding to particle type [[1, 1], [1, 2], [1, 3]]), the total number of particles is 10671+2678+875; After optimization iteration, the output is [1.009, 0.7915, 0.1, 3.3762, 1.1616, 0.3171, 51.6997, 1, 1, 1];
For particles with a charge number of 2 (corresponding to particle type [[2, 3], [2, 4]]), the total number of particles is 867+4762; After optimization iteration, the output is [1.418, 0.7085, 0.1, 7.6019, 0.9271, 0.4643, 29.7271, 1, 1];
For particles with a charge number of 3 (corresponding to particle type [[3, 6], [3, 7]]), the total number of particles is 525+210; After optimization iteration, the output is [1.0485, 0.7156, 1.4478, 3.0988, 1.2939, 0.9243, 69.4085, 1, 1];
For particles with a charge number of 4 (corresponding to particle type [[4, 9]]), the total number of particles is 180; After optimization iteration, the output is [0.9369, 0.6325, 1.3739, 11.6925, 0.5317, 0.693, 42.4851, 1];
For particles with a charge number of 5 (corresponding to particle type [[5, 10], [5, 11]]), the total number of particles is 297+183; After optimization iteration, the output is [1.0054, 0.7096, 1.8058, 6.1572, 1.122, 0.6146, 78.7048, 1, 1];
The visualization of the constructed particle identification curve set is shown in FIG. 3, in the figure: the curve formed by particles with the same charge number and mass number (represented by scatter points in the figure) is a particle identification curve; The isotope particle identification curves, formed by particles with the same charge number but different mass numbers, constitute a group of curves; The charge numbers of the particle identification curve groups are arranged in increasing order from bottom to top, and within each group of particle identification curves, the mass numbers are arranged in increasing order from bottom to top;
S3) Clean the input data of the particles to be identified that are produced by current medium-low energy heavy ion nuclear reactions, and extract only the collected two-dimensional energy spectrum feature data E and ΔE for each particle;
S4) Substitute the collected data of E corresponding to the particle j to be identified into formula (1) described in step S2), and sequentially calculate the ΔE values using the particle identification curve parameters and charge number and mass number corresponding to each particle identification curve in the particle identification curve set constructed in step S2), and denote the calculated ΔE value as ΔEⱼ; the absolute difference between ΔE (the original collected value) and ΔEⱼ (the calculated value) is taken as the distance from the particle j to the corresponding particle identification curve, and the charge number and mass number of the particle identification curve that results in the minimum absolute value of ΔE−ΔEⱼ are determined to be the particle type for particle j;
S5) Save the outlier particle data whose distance to every particle identification curve in the particle identification curve set exceeds the preset threshold (adjustable by the user according to the required precision); After reaching the preset quantity (determined by the user according to actual requirements, typically at least 1,000), re-execute Step S2) with the original data to update the particle identification curve set.

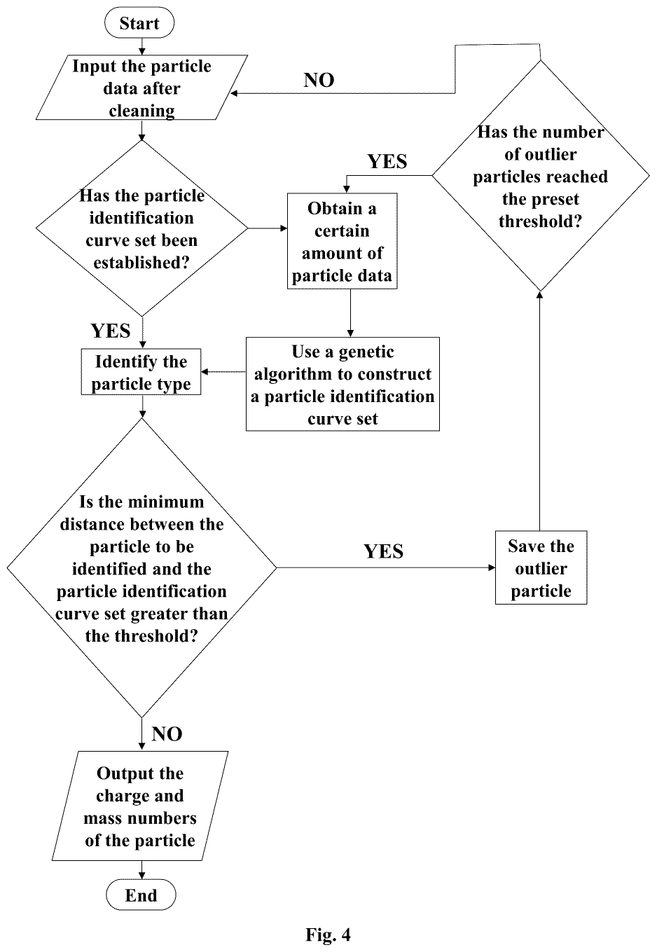

FIG. 4 is a flowchart illustrating the steps executed by computer for the method of intelligent identification of particle types produced by medium-low energy heavy ion nuclear reactions according to the present invention.

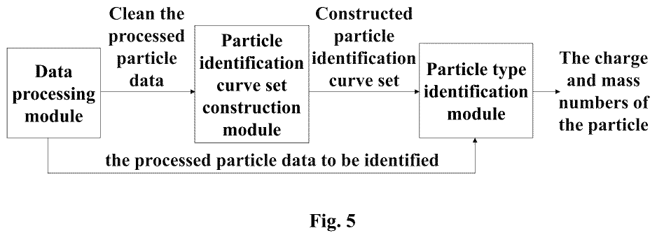

FIG. 5 is a structural block diagram of a system provided by an embodiment of the present invention for intelligent identification of particle types produced by medium-low energy heavy ion nuclear reactions, as shown in FIG. 5, the system includes:
Data processing module, which is used for cleaning and processing the raw data of the particles produced by medium-low energy heavy ion nuclear reaction, in order to extract the two-dimensional energy spectrum features E and ΔE of the particles;
Particle identification curve set construction module, which is used to create a particle library and construct a particle identification curve set using a genetic algorithm;
Particle type identification module, which is used to identify the particle types produced by medium-low energy heavy ion nuclear reaction.

The above functional modules generally include routines, programs, objects, components, data structures, etc., which perform specific tasks or implement specific abstract data types. These modules can exist physically as separate entities, or two or more modules can be integrated into one. These modules can be implemented in hardware form or in the form of software functional modules. If the physical or integrated modules are implemented as software functional modules and sold or used as independent products, they can also be stored in a computer-readable storage medium.

The present invention also provides a storage medium, which stores one or more programs with execution instructions. These execution instructions can be read and executed by electronic devices (including but not limited to computers, servers, network devices, etc.) to implement the method for intelligent identification of particle types produced by medium-low energy heavy ion nuclear reactions described above.

The present invention also provides an electronic device, which comprising:
at least one processor; and
a memory communicatively coupled to the at least one processor and storing program instructions executable by the at least one processor;
wherein the program instructions, when executed by the at least one processor, can cause the at least one processor to perform the method for intelligent identification of particle types produced by medium-low energy heavy ion nuclear reactions described above.

The memory may include a storage area for programs and a storage area for data. The program storage area can store the operating system and at least one application required by the functionality. The data storage area can store data created by the system for intelligent identification of particle types produced by medium-low energy heavy ion nuclear reactions described above. Additionally, the memory may include high-speed random-access memory (RAM), and may also include non-volatile memory, such as at least one disk storage device, flash memory device, or other non-volatile solid-state storage devices. In some examples, the memory may optionally include remote storage located away from the processor. These remote storages can be connected to the system for intelligent identification of particle types via a network. The network includes, but is not limited to, the Internet, intranets, local area networks (LAN), mobile communication networks, and their combinations.

The electronic device of the present invention can exist in various forms, including but not limited to:
(1) Mobile communication devices: These devices are characterized by their mobile communication capabilities and are primarily aimed at providing voice and data communication; These terminals include smartphones (e.g., iPhone), multimedia phones, feature phones, and low-end phones;
(2) Ultra-mobile personal computing devices: These devices belong to the type of personal computers, with computing and processing functions, and generally have mobile internet capabilities; These terminals include PDAs, MIDs, and UMPC devices, such as the iPad;
(3) Portable entertainment devices: These devices can display and play multimedia content, such devices include audio and video players (e.g., iPod), handheld game consoles, e-readers, as well as smart toys and portable in-vehicle navigation devices;
(4) Servers: Devices that provide computing services, a server is composed of components such as the processor, hard disk, memory, and system bus; Although similar to general computer architecture, servers require high reliability and have higher demands for processing power, stability, reliability, security, scalability, and manageability;
(5) Other electronic devices with data interaction capabilities.

The invention also provides a computer program product, which includes a computer program stored on a storage medium. The program contains instructions that, when executed by a computer, enable the computer to perform the method for intelligent identification of particle types produced by medium-low energy heavy ion nuclear reactions as described above. This computer software product can be stored in a computer-readable storage medium, such as ROM/RAM, disks, optical disks, etc., and includes several instructions that enable a computer device (which can be a personal computer, server, or network device) to execute the method described in the examples or certain portions of the method.

In addition, the logic and/or steps represented in the flowchart or otherwise described herein, such as executable instructions for implementing logical functions, can specifically be implemented in any computer-readable medium for use by a system, device, or equipment (such as a computer-based system, a system including a processor, or other systems capable of fetching and executing instructions from a system, device, or equipment). For the purposes of this description, “computer-readable media” can refer to any device that can contain, store, communicate, propagate, or transmit programs for use by an instruction-executing system, device, or equipment, or in combination with those instruction-executing systems, devices, or equipment. More specific examples of computer-readable media (non-exhaustive list) include: electronic devices with one or more wiring components, portable computer disk drives (magnetic devices), random access memory (RAM), read-only memory (ROM), erasable programmable read-only memory (EPROM or flash memory), optical fiber devices, and portable compact disk read-only memory (CD-ROM). Additionally, computer-readable media may even be paper or other suitable media, on which the program can be printed, since the program can be scanned from the paper or other medium, edited, interpreted, or otherwise processed as necessary, and then electronically stored in a computer memory.

It should be understood that the various parts of the invention can be implemented using hardware, software, firmware, or a combination thereof. In the examples above, several steps or methods can be implemented through software or firmware stored in a memory and executed by an appropriate instruction-executing system. Those skilled in the art will understand that all or part of the steps of the method in the examples can be completed using hardware associated with relevant instructions, and such programs can be stored in a computer-readable storage medium, with execution including any combination of the steps described in the method examples.

Finally, it should be noted that the above are only specific examples of the invention, and the protection scope of the invention is not limited thereto. Any changes or substitutions within the technical scope disclosed in the invention that would be easily conceived by those skilled in the art should fall within the protection scope of the invention. Where there are no conflicts, the examples and features in the examples in this application can be combined. Additionally, the method examples described above are presented as a series of actions for simplicity, but those skilled in the art should understand that the invention is not restricted by the described sequence of actions, as certain steps can be performed in other orders or simultaneously. Furthermore, those skilled in the art should understand that the examples described in the specification are preferred examples.

1. A method for intelligent identification of particle types produced by medium-low energy heavy ion nuclear reactions, comprising the following steps:
S1) creating a particle library containing all possible particle types that can be produced by current medium-low energy heavy ion nuclear reactions, where the particle library is a nested array consisting of arrays representing particle types by their charge number and mass number;
S2) using an algorithm and the particle library created in step S1) to construct a set of particle identification curves, where the particle identification curves conform to the following formula (1):
$$ΔE = {[ {( gE )}^{μ + v + 1} + {( λ {Z}^{α} {A}^{β} )}^{μ + v + 1} + ξ {Z}^{2} {{A}^{μ} ( g E )}^{v} ]}^{( \frac{1}{μ + v + 1} )} - gE ; \qquad ( 1 )$$
where E and ΔE are the two-dimensional energy spectrum feature values of the particles, Z is the charge number of the particle, A is the mass number of the particle, and g, λ, ζ, α, β, μ and v are the parameters of the particle identification curve, all the parameters of the particle identification curve are greater than 0 and are iteratively optimized using the genetic algorithm;
the particle identification curve set being a collection of multiple particle identification curves formed by substituting the particle identification curve parameters and their corresponding particle type charge numbers and mass numbers into formula (1);
S3) cleaning input data of the particles to be identified that are produced by current medium-low energy heavy ion nuclear reactions, and extracting only the collected two-dimensional energy spectrum feature data E and ΔE for each particle; and
S4) substituting the collected data of E corresponding to the particle j to be identified into formula (1) described in step S2), and sequentially calculating the ΔE values using the particle identification curve parameters and charge number and mass number corresponding to each particle identification curve in the particle identification curve set constructed in step S2), and denote the calculated ΔE value as ΔEⱼ the absolute difference between ΔE (the original collected value) and ΔEⱼ (the calculated value) being taken as the distance from the particle j to the corresponding particle identification curve, and the charge number and mass number of the particle identification curve that results in the minimum absolute value of ΔE−ΔEⱼ being determined to be the type for particle j.

2. The method according to claim 1, wherein the process of constructing the particle identification curve set in step S2) includes the following specific steps:
S21) obtaining an existing particle dataset for a reaction identical to the current medium-low energy heavy ion nuclear reaction, cleaning the data to remove redundant features, and extracting the required two-dimensional energy spectrum feature data E and ΔE for each particle;
S22) randomly generating a certain number of individuals, whose collection is referred to as the population; each individual is composed of the particle identification curve parameters g, λ, ζ, α, β, μ, v and an integer variable Nᵢ to determine whether the particle type in the particle library exists in the obtained existing dataset, the value of i corresponds to the sequence number of the particle type in the particle library, starting from 1, and the value of Nᵢ is either 0 or 1; if Nᵢ is 0, then the particle type does not exist in the dataset; if Nᵢ is 1, then the particle type exists in the dataset; the initial values of g, λ, ζ, α, β, μ, v and Nᵢ for each individual are all randomly generated by the system;
S23) calculating the fitness value y for each individual using the fitness function shown in the following formula (2):
$$y = 10 \; 0 * \frac{1}{N l} * {\sum }_{i = 1}^{Nl} \frac{{\sum }_{j = 1}^{{Np}_{i}} {( Δ E - Δ {E}_{j} )}^{2}}{{Np}_{i}} ; \qquad ( 2 )$$
where Nl represents the number of curves, Npᵢ represents the number of particles corresponding to the i-th curve, ΔEⱼ represents the ΔE calculated for particle j using formula (1), and ΔE represents the original collected value for particle j; Additionally, Npᵢ is not 0, Nl refers to the number of curves corresponding to particle type where Nᵢ is 1 and Npᵢ is not 0; the value of ΔE−ΔEⱼ is the minimum distance between particle j and the curves corresponding to particle type where Nᵢ is 1;
S24) based on the calculated fitness value y for each individual, selecting suitable individuals from the population to generate the next generation;
S25) adding the generated next generation to the population by replacement or direct addition;
S26) repeating steps S21) to S25) until the iteration stopping conditions are met;
S27) after the iteration is completed, outputting the individual with the smallest fitness value y;
S28) combining the charge number and mass number of the particle type where Nᵢ=1 and Npᵢ≠0 with the particle identification curve parameters g, λ, ζ, α, β, μ and v from the output individual, and substitute them into formula (1) to obtain the particle identification curve set.

3. The method according to claim 2, wherein step S2) further includes the following optimization iteration step:
S29) grouping the particles in the constructed particle identification curve set by charge number, and grouping particles with the same charge number together; then performing steps S21) to S28) for each group of particles to optimize the iteration.

4. The method according to claim 2, wherein the step S23) includes the following specific steps:
S231) traversing the existing particle dataset obtained in step S21);
S232) obtaining the two-dimensional energy spectrum feature data E and ΔE for particle j;
S233) for each individual, sequentially calculating the values of ΔE−ΔEⱼ using the particle identification curve parameters and the charge number and mass number of the particle type where Nᵢ is 1, select the minimum absolute value and increment Npi for the particle type corresponding to the smallest absolute value to count the number of particles of that type; and
S234) continuing to obtain the next particle's two-dimensional energy spectrum feature data E and ΔE and repeat the step S233).

5. The method according to claim 2, wherein step S24) includes the following specific steps:
S241) Selection: Based on the calculated fitness value y for each individual, selecting individuals with relatively smaller fitness values as the next generation;
S242) Mutation: Randomly modifying the data features of some individuals in the population to prevent excessive similarity between individuals, thus promoting population diversity;
S243) Crossover: performing random crossover combinations of some individuals in the population to generate new individuals.

6. The method according to claim 1, wherein the method further includes the following step:
S5) saving the outlier particle data whose distance to every particle identification curve in the particle identification curve set exceeds the preset threshold; after reaching the preset quantity, re-execute Step S2) with the original data to update the particle identification curve set.

7. A system for intelligent identification of particle types produced by medium-low energy heavy ion nuclear reactions, comprising:
a data processing module configured to clean and process the raw data of the particles produced by medium-low energy heavy ion nuclear reactions, in order to extract the two-dimensional energy spectrum features E and ΔE of the particles;
a particle identification curve set construction module configured to create a particle library and construct a particle identification curve set using a genetic algorithm; and
a particle type identification module configured to identify the particle types produced by medium-low energy heavy ion nuclear reactions.

8. A storage medium, on which a computer program is stored, wherein the method according to claim 1 is realized when the program is executed by the processor.

9. An electronic device, comprising:
at least one processor; and
a memory communicatively coupled to the at least one processor and storing program instructions executable by the at least one processor;
wherein the program instructions, when executed by the at least one processor, cause the at least one processor to perform the method according to claim 1.

10. A computer program product, comprising a computer program stored on a storage medium, the computer program comprising program instructions, wherein, when the program instructions are executed by a processor, the processor configured to perform the method according to claim 1.
